SEQUENCE LISTING (8 sequences)

SEQ ID NO: 1 - DNA, 1748 bp, synthetic construct
  source 1..1748: mol_type = other DNA
ttcttagcggccgccaccatggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacaggtaagaatcaagcaaacttcattaataatttatgttcttatgttgacagttcttttgtgattatactcattatttattacacactttttctcacctacagcccaagagtgttcgctggatgatgacaccattctaatcccaattatagttggtgctggtctttcaggcttgattatcgttatagtgattgcttacgtaattggcagaagaaaaagttatgctggatatcagactctgtaattgaaagtttgtcactgtttctgttctcaattgtcctttctccacatctagctcaagactgcagtgcagatgacgacaacttccttgtgcccatagcggtgggagctgccttggcaggagtacttattctagtgttgctggcttattttattggtctcaagcaccatcatgctggatatgagcaattttagattgcattctttgctaatttcctttccttgctgtctgttcttttctaacagctgaagaatgttctgctgactctgacctcaactttcttattcctgttgcagtgggtgtggccttgggcttccttataattgttgtctttatctcttatatgattggaagaaggaaaagtcgtactggttatcagtctgtgtaagcatgcattctt

SEQ ID NO: 2 - DNA, 2245 bp, synthetic construct
  source 1..2245: mol_type = other DNA
ttcttagcggccgccaccatggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacaggtaagaatcaagcaaacttcattaataatttatgttcttatgttgacaggtggcttttcctttgaatcatagacatttaaacgttgaaatagaggaatatgaattcatagccttgaccaaattgtgcaactctatatttggctcatcattttgctcaaccgagatgtttttgtgatcttaaaatcatttgcaaaaaaagttcttaaagctaaaactgatagaagctttaagtgagctttatttgaggtatgggttttagtttgtagggaagatggcacttgggagcagcagctttgagcatcttgcatctgcttcctgaggccgtcttctcactggaaacattttcatacctgaatgaaagcagtttggtctgattttgttaagacaaaagataaagagcggtagcaaagcctcaaatatcatgaactatgtttttaccagccaaaacgatctggagagttttcaaagcatgtgccagtgtgaatgacgctttttgaagatcactcagggataatggtggtatttattagatttttaatgaaaccttcttttgtgattatactcattatttattacacactttttctcacctacagcccaagagtgttcgctggatgatgacaccattctaatcccaattatagttggtgctggtctttcaggcttgattatcgttatagtgattgcttacgtaattggcagaagaaaaagttatgctggatatcagactctgtaattgaaagtttgtcactgtttctgttctcaattgtcctttctccacatctagctcaagactgcagtgcagatgacgacaacttccttgtgcccatagcggtgggagctgccttggcaggagtacttattctagtgttgctggcttattttattggtctcaagcaccatcatgctggatatgagcaattttagattgcattctttgctaatttcctttccttgctgtctgttcttttctaacagctgaagaatgttctgctgactctgacctcaactttcttattcctgttgcagtgggtgtggccttgggcttccttataattgttgtctttatctcttatatgattggaagaaggaaaagtcgtactggttatcagtctgtgtaatgcatgcattctt

SEQ ID NO: 3 - DNA, 2237 bp, synthetic construct
  source 1..2237: mol_type = other DNA
ttcttagcggccgccaccatggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacaggtaagaatcaagcaaacttcattaataatttatgttcttatgttgacagttcttttgtgattatactcattatttattacacactttttctcacctacagcccaagagtgttcgctggatgatgacaccattctaatcccaattatagttggtgctggtctttcaggcttgattatcgttatagtgattgcttacgtaattggcagaagaaaaagttatgctggatatcagactctgtaacactagactgagcatcttgaggcaaacagttatgtcatcagggcccacttatatctcttatatctcttttcccattagtaaatgatcccagattgtaatgtattgagttcttatgtctgctttgtatagttgttcattcagtgagttaatacttaacagcaatatttgaagcattgtgcttcttcgacatcataacagtttgcctctgaaagctcacggcctgatcatcaaggcaaggctaattagatttgttcattatgttctgtttaagaaatctgggaactatgtgaccatgttatacatttttcatccaaaatgactgcatttttacttggtacttaaaagtgtattatttggttatttggaaaatcacttttgtggaacatctcatttgctgataatgaaatgccaatgacttgttaccttggtataaaaaaccaaaatatgcgtgttaggaagtgctgtcattttactcaaaatacacagatttgaaagtttgtcactgtttctgttctcaattgtcctttctccacatctagctcaagactgcagtgcagatgacgacaacttccttgtgcccatagcggtgggagctgccttggcaggagtacttattctagtgttgctggcttattttattggtctcaagcaccatcatgctggatatgagcaattttagattgcattctttgctaatttcctttccttgctgtctgttcttttctaacagctgaagaatgttctgctgactctgacctcaactttcttattcctgttgcagtgggtgtggccttgggcttccttataattgttgtctttatctcttatatgattggaagaaggaaaagtcgtactggttatcagtctgtgtaatgcatgcattctt

SEQ ID NO: 4 - DNA, 2271 bp, synthetic construct
  source 1..2271: mol_type = other DNA
ttcttagcggccgccaccatggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacaggtaagaatcaagcaaacttcattaataatttatgttcttatgttgacaggtggcttttcctttgaatcatagacatttaaacgttgaaatagaggaatatgaattcatagccttgaccaaattgtgcaactctatatttggctcatcattttgctcaaccgagatgtttttgtgatcttaaaatcatttgcaaaaaaagttttaattctagaacagttctccattttctatagctttatcataaaaatatacctgtcttagttacttgaagttgatcttgtaagaacatttctgacttttaaatcaacatctttgacttcgagacattcttttgtgattatactcattatttattacacactttttctcacctacagcccaagagtgttcgctggatgatgacaccattctaatcccaattatagttggtgctggtctttcaggcttgattatcgttatagtgattgcttacgtaattggcagaagaaaaagttatgctggatatcagactctgtaacacttttgtggaacatctcatttgctgataatgaaatgccaatgacttgttaccttggtataaaaaaccaaaatatgcgtgttaggaagtgctgtcattttactcaaaatacacagatttgaaagtttgtcactgtttctgttctcaattgtcctttctccacatctagctcaagactgcagtgcagatgacgacaacttccttgtgcccatagcggtgggagctgccttggcaggagtacttattctagtgttgctggcttattttattggtctcaagcaccatcatgctggatatgagcaattttagaatttcgtgtgattttagataaagtgataatgagatattgagcaaataagaaaatagaggtaatgctataaaaaactaagctatgtacactttcaaaatgcatgtttcttgcatgtttttactacttaattgcattctttgctaatttcctttccttgctgtctgttcttttctaacagctgaagaatgttctgctgactctgacctcaactttcttattcctgttgcagtgggtgtggccttgggcttccttataattgttgtctttatctcttatatgattggaagaaggaaaagtcgtactggttatcagtctgtgtaagcatgcattctt

SEQ ID NO: 5 - DNA, 1555 bp, synthetic construct
  source 1..1555: mol_type = other DNA
ttcttagcggccgccaccatggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacaggtaagaatcaagcaaacttcattaataatttatgttcttatgttgacagttcttttgtgattatactcattatttattacacactttttctcacctacagcccaagagtgttcgctggatgatgacaccattctaatcccaattatagttggtgctggtctttcaggcttgattatcgttatagtgattgcttacgtaattggcagaagaaaaagttatgctggatatcagactctgtaatttgaaagtttgtcactgtttctgttctcaattgtcctttctccacatctagctcaagactgcagtgcagatgacgacaacttccttgtgcccatagcggtgggagctgccttggcaggagtacttattctagtgttgctggcttattttattggtctcaagcaccatcatgctggatatgagcaattttaggcatgcattctt

SEQ ID NO: 6 - DNA, 1554 bp, synthetic construct
  source 1..1554: mol_type = other DNA
ttcttagcggccgccaccatggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacaggtaagaatcaagcaaacttcattaataatttatgttcttatgttgacagttcttttgtgattatactcattatttattacacactttttctcacctacagcccaagagtgttcgctggatgatgacaccattctaatcccaattatagttggtgctggtctttcaggcttgattatcgttatagtgattgcttacgtaattggcagaagaaaaagttatgctggatatcagactctgtaattcttttgtgattatactcattatttattacacactttttctcacctacagctcaagactgcagtgcagatgacgacaacttccttgtgcccatagcggtgggagctgccttggcaggagtacttattctagtgttgctggcttattttattggtctcaagcaccatcatgctggatatgagcaattttaggcatgcattctt

SEQ ID NO: 7 - DNA, 1554 bp, synthetic construct
  source 1..1554: mol_type = other DNA
ttcttagcggccgccaccatggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacaggtaagaatcaagcaaacttcattaataatttatgttcttatgttgacagttcttttgtgattatactcattatttattacacactttttctcacctacagcccaagagtgttcgctggatgatgacaccattctaatcccaattatagttggtgctggtctttcaggcttgattatcgttatagtgattgcttacgtaattggcagaagaaaaagttatgctggatatcagactctgtaaattgcattctttgctaatttcctttccttgctgtctgttcttttctaacagctcaagactgcagtgcagatgacgacaacttccttgtgcccatagcggtgggagctgccttggcaggagtacttattctagtgttgctggcttattttattggtctcaagcaccatcatgctggatatgagcaattttaggcatgcattctt

SEQ ID NO: 8 - DNA, 2381 bp, synthetic construct
  source 1..2381: mol_type = other DNA
gtcgaccagaaataccatttgacgcatgcacacgtatgtttattgcagcactatttacaatagcaaagacttggaaccaacccaaatgcccatcaatgatagactagataaagaaaacgtggcacatatatgccatggaatactatgcagccataaaaaaatgagatcatgtcctttgcagggacatggatgaagctggaagccatcatccttagcaaactaacacaggaacagaaaacaaaacactgcatgttctcactcataagtgggagttgaacaatgagaacacatggacacagggaggggaacaacacacacgggggcctgtcaaggggtgggggccaaggggagggagagcattaggagaaatacctaatgcatgtggggcttaaaacctagatgacgggttgataggtgcagtaaaccaccatggcacatgtatacctatgtaacaaagctgcacattctgcacatgtatcccagaacttaaagtaaaattaaaaaaaaaaaaaaaaaagaaagaaaaagaaaaagaaaatgccgtccgcgcatatctctcaggagcataggaagggctgtgaaccaaaaagccagggcgtggtcgccccgcccaccggccgctacatcaccacccctctcgccgctttcaagcgcggagattggctgtaagcaagaaagagccccgcccctagtcttatgactcgcactgaagcgccgattcctggcttttgcaaggctgtggtcggtggtcatcagtgctcttgacccaggtccagcgagccttttccctggtgttgcagctgttgttgtaccgccgccgtcgccgccgtcgccgcctgctctgcggggtcatggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacaggtaagaatcaagcaaacttcattaataatttatgttcttatgttgacagttcttttgtgattatactcattatttattacacactttttctcacctacagcccaagagtgttcgctggatgatgacaccattctaatcccaattatagttggtgctggtctttcaggcttgattatcgttatagtgattgcttacgtaattggcagaagaaaaagttatgctggatatcagactctgtaattcttttgtgattatactcattatttattacacactttttctcacctacagctcaagactgcagtgcagatgacgacaacttccttgtgcccatagcggtgggagctgccttggcaggagtacttattctagtgttgctggcttattttattggtctcaagcaccatcatgctggatatgagcaattttaggcatgc

(19) United States
(12) Patent Application Publication
(10) Pub. No.: US 2026/0224744 A1
(43) Pub. Date: Aug. 6, 2026

(54) GENE THERAPY TO TREAT LAMP2 ASSOCIATED RETINOPATHY IN DANON DISEASE

(71) Applicant: The Regents of the University of California, Oakland, CA (US)

(72) Inventors: Shyamanga Borooah, San Diego, CA (US); Eric Adler, La Jolla, CA (US); Paul J. Bushway, San Diego, CA (US)

(21) Appl. No.: 19/145,837

(22) PCT Filed: Jan. 8, 2024
(86) PCT No.: PCT/US2024/010631

§ 371 Date: Jul. 3, 2025

Related U.S. Application Data
(60) Provisional application No. 63/478,711, filed on Jan. 6, 2023.

(51) Int. Cl. A61K 48/00; A61K 38/17; A61P 27/02; C12N 15/86

(52) U.S. Cl. CPC A61K 48/005; A61K 38/177; A61P 27/02; C12N 15/86; C12N 2750/14143

(57) ABSTRACT

Gene therapies and methods for treating a disease or condition characterized by LAMP2 deficiency in a patient, such as retinal disease and/or degeneration in Danon disease. A method for treating retinal disease in a patient comprising administering to the patient an effective amount of a vector for expressing two or more lysosome associated membrane protein 2 (LAMP2) isoforms in a cell of the patient. A composition for treating retinal disease in a patient comprising a nucleotide vector for expressing two or more lysosome associated membrane protein 2 (LAMP2) isoforms.

### CROSS-REFERENCE TO RELATED APPLICATIONS

This application claims priority to U.S. Provisional Application No. 63/478,711, filed on Jan. 6, 2023, the entire contents of which are incorporated by reference.

### SEQUENCE LISTING

The instant application contains a Sequence Listing which has been submitted electronically and is hereby incorporated by reference in its entirety. Said electronic copy, created on ______, is named ______ and is ______ bytes in size.

### TECHNICAL FIELD

The present invention relates to gene therapies for treating retinal disease and/or degeneration associated with Danon disease.

### BACKGROUND

Danon disease is an X-linked disorder caused by mutations in the gene, lysosome-associated membrane 2 (LAMP2). Danon disease is a systemic disease with severe ocular manifestations. Danon disease affects patients from childhood resulting in hypertrophic cardiomyopathy, arrythmias, skeletal muscle myopathy, and intellectual disability. Affected children describe nyctalopia and blurry vision. Retinal examination reveals findings which include pigmentary retinopathy and peripheral ‘salt-and-pepper’ retinopathy. Electrophysiological studies show a cone-rod dystrophy and reduced electrooculographic readings. Both males and carrier females are affected by retinopathy although there is marked inter individual variation. Although progress has been made in the understanding and management of cardiac disease in Danon disease, knowledge of the cause of the ocular disease and the development of treatments for retinal disease has been limited by a lack of models.

LAMP2 is expressed throughout the body and in the retinal pigment epithelium (RPE) and to a lesser extent the neuroretina within the eye. LAMP2 is commonly found on the membranes of cellular lysosomes. Three different isoforms of LAMP2 are known: LAMP2A, LAMP2B, and LAMP2C. Expression of the isoforms varies between organs and species. The expression profile in the human eye has not yet been established although it appears, from studies in ARPE-19 cell line and mice, that LAMP2A and LAMP2B are the predominant forms expressed in the eye.

Dysregulated autophagy is the predominant cause of the cardiac manifestations of Danon disease. Each of the LAMP2 isoforms is thought to play differing roles in autophagy. Three are three main forms of autophagy; chaperone mediated, micro and macro autophagy. Macroautophagy is most affected in cardiac disease in Danon disease. LAMP2B is the isoform predominantly associated with macroautophagy leaving chaperone mediated autophagy largely unaffected. These findings have been supported by recent cardiologic studies using human induced pluripotent stem cells (hiPSCs) differentiated to cardiomyocytes which found CRISPR-Cas knockout of LAMP2B recapitulated many of the findings seen in cardiomyocytes generated from Danon disease patients and human post-mortem cardiac samples. LAMP2B knockout, resulted in decreased clearance of autophagic vacuoles and an increase in LC3-II. LC3 is an essential protein for autophagosome formation. The lipidated form of LC3 is called LC3-II. LC3-II is present in all autophagic vacuoles. LAMP2B was shown to be important for lysosomal binding to LC3-II positive autophagosomes generating lipophagosomes. Defects downstream of lysosomal fusion lead to increased LC3-II positive autophagic vacuoles and are a good marker for dysregulated liposomal fusion.

The cause of retinopathy in Danon disease is still incompletely understood. RPE cells seem to be the primary cell affected in Danon disease retinopathy. A number of studies have shown that autophagy occurs in RPE. RPE may be particularly susceptible to dysfunctional autophagy as they are exposed to oxidative stress which increases autophagic flux. Recent studies link phagocytosis to the autophagic pathway. Photoreceptor outer segment (POS) containing phagosomes were shown to be LC3-II lined. Additionally, LAMP-2 is highly expressed in RPE lysosomes which are required for ROS degradation. As a result the requirement for RPE to phagocytose and digest large numbers of POS may make RPE even more susceptible to damage resulting from dysfunctional autophagic pathways.

### SUMMARY OF THE INVENTION

Disclosed herein are compositions, gene therapies and methods for treating a disease or condition characterized by LAMP2 deficiency. Disclosed herein are compositions, gene therapies and methods for treating retinal disease and/or degeneration in Danon disease, particularly LAMP2 associated retinopathy.

In an aspect, a method for treating a disease or condition characterized by LAMP2 deficiency in a patient is provided, the method comprising administering to the patient an effective amount of a vector for expressing two or more lysosome associated membrane protein 2 (LAMP2) isoforms in a cell of the patient. In embodiments, the two or more LAMP2 isoforms are selected from LAMP2A, LAMP2B and/or LAMP2C isoforms.

In an aspect, a method for treating retinal disease in a patient is provided, the method comprising administering to the patient an effective amount of a vector for expressing two or more lysosome associated membrane protein 2 (LAMP2) isoforms in a cell of the subject. In some embodiments, the treatment may target neuroretinal and retinal pigment epithelium (RPE) cells to express LAMP2. In embodiments, the vector is administered by injection into the vitreous, the suprachoroid or under the retina of the patient. In embodiments, the treatment restores retinal function and/or preserves or improves vision in the patient. In embodiments, the retinal disease is Danon disease. In embodiments, the retinal disease is age-related macular degeneration.

In another aspect, a composition for treating retinal disease in a patient is provided, the method comprising a nucleotide vector for expressing two or more lysosome associated membrane protein 2 (LAMP2) isoforms in a cell of the patient. In some embodiments, the vector is an adeno-associated virus expressing the two or more LAMP2 isoforms. In some embodiments, the LAMP2 isoforms are selected from LAMP2A, LAMP2B and/or LAMP2C isoforms.

In a further aspect, a neuroretinal cell or retinal pigment epithelium (RPE) cell is provided, where the cell is modified to express two or more LAMP2 isoforms. In some embodiments, the LAMP2 isoforms are selected from LAMP2A, LAMP2B and/or LAMP2C isoforms.

### BRIEF DESCRIPTION OF THE DRAWINGS

FIG. 1. Imaging of Danon retinopathy. Panel A shows optos multicolor imaging from a 15 year old female carrier showing a region of peripheral RPE atrophy (white arrowheads). Panel B shows optos scanning laser ophthalmoscopy in the same 15 year old female carrier showing increased peripheral discrete hyperautofluorescence (white arrowheads). Panel C shows spectral domain optical coherence tomography B scan from a 10 year old male case. Panel C′ shows an enlarged image from square in Panel C demonstrating disrupted outer nuclear layer (white arrowheads).

FIGS. 2A-2C. Mouse retinal studies. FIG. 2A. Scanning laser ophthalmoscopy showing multiple discrete hyperautofluorescent spots in an 18 month old LMAP2 KO mouse. FIG. 2B. Spectral domain optical coherence tomography B-scan from an 18 year old mouse showing deposits disrupting the outer retina. FIG. 2C. Retinal cryosection from a 6 month old WT mouse showing LAMP2 immunostaining (Green greyscales) in the RPE and the IS/ONL boundary (white arrowheads).

FIG. 3. Retinal electrophysiology responses from 20-24 mo old WT (n=7) and 18-22 mo old Lamp2 KO mice (n=3) demonstrating significant reductions in scotopic and photopic responses to +1.09 CD.S/m2 stimulation. (***p<0.001, **p<0.01).

FIGS. 4A-4C. Mouse retinal sections. FIG. 4A. Retinal cryosection from a 22 month old wild type mouse stained with DAPI (blue) and S-Opsin (Green) (Scale=30 μm). FIG. 4B. Retinal cryosection form a 18 month old Lamp2 KO mouse showing the presence of many photoreceptors stained with DAPI (blue) and S-Opsin (Green) (Scale=30 m). FIG. 4C. Transmission electron microscopy section from a 22 month old Lamp2 KO mouse showing RPE and overlying outer segment.

FIG. 5. Hypothetical pathway demonstrating how LC3-II lined autophagosomes fuse with LAMP2B and lysosomes in order to degrade POS. Loss of LAMP2B should lead to increased LC3-II phagosomes in RPE.

FIGS. 6A-6G. Schematic representation of LAMP2 synthetic hnRNA sequences SynV1-SynV7. FIG. 6A. LAMP2 ABC SynV1. FIG. 6B. LAMP2 ABC SynV2. FIG. 6C. LAMP2 ABC SynV3. FIG. 6D. LAMP2 ABC SynV4. FIG. 6E. LAMP2 ABC SynV5. FIG. 6F. LAMP2 ABC SynV6. FIG. 6G. LAMP2 ABC SynV7.

FIG. 7. Schematic representation of AAV-Promoter-LAMP2AC sequence.

FIGS. 8A-8C. Ectopic Expression of AAV.L2ABC SynV1 in Danon iPSCs. FIGS. 8A-8B. Comparison of EF1 (FIG. 8A) and PGK1 (FIG. 8B) promoters. FIG. 8C. Western blot protein quantification of EF1 and PGK1 promoters.

FIGS. 9A-9B. Sequential qPCR on EF1-L2ABC SynV1-V7. Dn iPS cells transfected with AAV plasmid (FIG. 9A) or control (FIG. 9B) cells.

FIGS. 10A-10B. EF1-LAMP2ABC Protein Expression. FIG. 10A. Western blot of Danon iPS cells transfected with AAV plasmid or control cells. FIG. 10B. Quantification of Canon iPS cells transfected with AAV plasmid or control cells.

FIGS. 11A-11B. iPSC Danon Transfection. FIG. 11A. Western blot of Danon iPS cell transfection. FIG. 11B. Quantification of Danon iPS cell transfection.

FIGS. 12A-12C. Promoter LAMP2 transcription and protein abundance. FIG. 12A. Western plot of Danon iPS cells transfected with promoters. FIG. 12B. Quantification of Danon iPS cell transfection with and without promoters. FIG. 12C. Western blot of Danon iPS cells transfected with promoters.

FIGS. 13A-13B. Promoter-Kozak Rebuilds. FIG. 13A. Western blot of Danon rebuild iPS cell transfection. FIG. 13B. Quantification of Danon rebuild iPS cell transfection.

FIG. 14. Schematic representation of LAMP2 promoter sequence driving LAMP2ABsynV6.

### DETAILED DESCRIPTION

Disclosed herein are gene therapies and method for treating for treating a disease or condition characterized by LAMP2 deficiency. Disclosed herein are compositions, gene therapies and methods for treating retinal disease and/or degeneration in Danon disease, particularly LAMP2 associated retinopathy.

In an aspect, a method for treating a disease or condition characterized by LAMP2 deficiency in a patient is provided, the method comprising administering to the patient an effective amount of a vector for expressing two or more lysosome associated membrane protein 2 (LAMP2) isoforms in a cell of the patient. In embodiments, the two or more LAMP2 isoforms are selected from LAMP2A, LAMP2B and/or LAMP2C isoforms.

In an aspect, the disease or condition characterized by LAMP2 deficiency is retinal disease and/or degeneration, particularly LAMP2 associated retinopathy.

In an aspect, a method for treating retinal disease in a patient is provided, the method comprising administering to the patient in need thereof an effective amount of a nucleotide vector encoding two or more lysosome associated membrane protein 2 (LAMP2) isoforms in a cell of the subject. In some embodiments, the treatment may target neuroretinal and retinal pigment epithelium (RPE) cells to express the LAMP2 isoforms. In other embodiments, the treatment may restore retinal function and/or preserve or improve vision in the patient. In some embodiments, the retinal disease is Danon disease. In other embodiments, the retinal disease is age-related macular degeneration.

In another aspect, a composition for treating retinal disease in a patient is provided, the method comprising a nucleotide vector for expressing lysosome associated membrane protein 2 (LAMP2). In some embodiments, the vector is an adeno-associated virus expressing LAMP2. In some embodiments, the two or more LAMP2 isoforms are elected from isoforms A, B, and/or C.

In a further aspect, a cell, such as neuroretinal cell or retinal pigment epithelium (RPE) cell is provided, where the cell is modified by a nucleic acid vector to express two or more LAMP2 isoforms.

Various further aspects and embodiments of the disclosure are provided by the following description. Before further describing various embodiments of the presently disclosed inventive concepts in more detail by way of exemplary description, examples, and results, it is to be understood that the presently disclosed inventive concepts are not limited in application to the details of methods and compositions as set forth in the following description. The presently disclosed inventive concepts are capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the presently disclosed inventive concepts may be practiced without these specific details. In other instances, features which are well known to persons of ordinary skill in the art have not been described in detail to avoid unnecessary complication of the description. All of the compositions and methods of production and application and use thereof disclosed herein can be made and executed without undue experimentation in light of the present disclosure.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention may employ conventional techniques of molecular biology (including recombinant techniques), microbiology, cell biology, biochemistry and immunology, which are within the skill of the art. Such techniques are explained fully in the literature, such as Molecular Cloning: A Laboratory Manual, second edition (Sambrook et al, 1989) Cold Spring Harbor Press; Oligonucleotide Synthesis (MJ. Gait, ed., 1984); Methods in Molecular Biology, Humana Press; Cell Biology: A Laboratory Notebook (J. E. Cellis, ed., 1998) Academic Press: Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and CC. Blackwell, eds.); Gene Transfer Vectors for Mammalian Cells (J. M. Miller and M. P. Calos, eds., 1987); Current Protocols in Molecular Biology (F. M. Ausubel et al, eds., 1987); PCR: The Polymerase Chain Reaction, (Mullis et al, eds., 1994): Current Protocols in Immunology (J. E. Coligan et al, eds., 1991); Short Protocols in Molecular Biology (Wiley and Sons, 1999); Immunobiology (CA. Janeway and P. Travers, 1997). Although any methods and materials similar or equivalent to those described herein can be used in the practice of the present invention, the exemplary methods, devices, and materials are described herein. For the purposes of the present disclosure, the following terms are defined below. Additional definitions are set forth throughout this disclosure.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, an adeno-associated virus vector, a pharmaceutical composition, and/or a method that “comprises” a list of elements (e.g., components, features, or steps) is not necessarily limited to only those elements (or components or steps), but may include other elements (or components or steps) not expressly listed or inherent to the adeno-associated virus vector, pharmaceutical composition and/or method. Reference throughout this specification to “one embodiment,” “an embodiment,” “a particular embodiment,” “a related embodiment,” “a certain embodiment,” “an additional embodiment.” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”. It is understood that aspects and embodiments of the invention described herein include “consisting” and/or “consisting essentially of” aspects and embodiments.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

It will be further understood that there are a number of values disclosed therein, and that each value is also herein disclosed as “about” that particular value in addition to the value itself. In embodiments, “about” can be used to mean, for example, a quantity, level, value, number, frequency, percentage, dimension, size, amount, weight or length that varies by as much as 15%, 10%, 9%, 8%, 7%, 6%, 5%, 4%, 3%. 2% or 1% to a reference quantity, level, value, number, frequency, percentage, dimension, size, amount, weight or length. In various embodiments, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

The section headings used herein are for organizational purposes only and are not to be construed as limiting the subject matter described.

### Compositions of Matter

In an aspect, the disclosure provides a composition for treating diseases or conditions characterized by LAMP2 deficiency, such as retinal disease in a patient comprising a nucleotide vector for expressing two or more lysosome associated membrane protein 2 (LAMP2) isoforms. In some aspects, the LAMP2 isoforms are selected from isoforms A, B, and/or C. In some aspects, the vector is an adeno-associated virus expressing LAMP2.

This composition may target neuroretinal and retinal pigment epithelium (RPE) cells to express LAMP 2. This composition may be effective to restore retinal function in the patient and/or preserve or improve vision in the patient. In some aspects, the retinal disease is Danon disease. In other aspects, the retinal disease is age-related macular degeneration.

In another aspect, a neuroretinal cell or retinal pigment epithelium (RPE) cell is provided, where the cell is modified to express LAMP2.

The term “nucleic acid” or “polynucleotide”, includes DNA and RNA such as genomic DNA, cDNA and mRNA, or combinations thereof. The nucleic acid may comprise, in addition to the sequence enabling the genetic modifications of the disclosure, further sequences such as those required for the transcription and/or translation of the nucleic acid enabling said genetic modifications. This may include a promoter, enhancer, transcription and/or translation initiation and/or termination sequences, selection markers, sequences protecting or directing the RNA and/or enabling the genetic modifications within the cell. The selection and combination of these sequences is within the knowledge of the person skilled in the art and may be selected in accordance with the cell the nucleic acid is intended for.

As used herein. “engineered” or “genetically modified” or “transformed” are used interchangeably, wherein a cell has been manipulated by means of molecular reprogramming of a genomic sequence (e.g. by insertion, deletion, or substitution). Said cells include the primary transformed cell and progeny derived therefrom without regard to the number of passages. Progeny may not be completely identical in nucleic acid content to a parent cell and may contain mutations. Mutant progeny that have the same function or biological activity as screened or selected for in the originally transformed cell are included herein.

The terms “exogenous” and “heterologous” are used herein to refer to any molecule, including nucleic acids, protein or peptides, small molecular compounds, and the like that originate from outside the organism. In contrast, the term “endogenous” refers to any molecule that originates from inside the organism (i.e., naturally produced by the organism).

In an aspect, the disclosure provides a purified cell composition comprising one or more of the nucleotide vector of the disclosure.

In some embodiments, at least about 50%, 60%, 70%, 80%, 90%, or 100% of the nucleotide vector treated cells expresses LAMP2.

In some embodiments, the nucleotide vector treated cells have increased expression of LAMP2 as compared to non-nucleotide vector treated cells. In some embodiments the disease cell line is a cell line comprising a malignancy. In some embodiments the disease cell line is a cell line comprising a viral infection.

### Method of Making

The disclosure provides a method of making a nucleotide vector.

Genome editing tools may be used to manipulate protein expression of certain cells and/or cell types. In some embodiments, LAMP2 expression may be manipulated with nucleic acid vectors, including viral vectors and other genome editing tools, as disclosed herein.

Polynucleotides enabling the genetic modifications of the disclosure may be delivered to cells as an isolated nucleic acid or in a vector. The isolated nucleic acid or the vector may be delivered in lipid- or lipid-based delivery system, such as a liposome. Alternatively, the vector may comprise viral proteins, such as when the vector is a viral vector. The term “vector” as used herein refers to a construction comprised of genetic material designed to direct transformation or transductions of a targeted cell. A vector contains multiple genetic elements positionally and sequentially oriented with other necessary elements such that the nucleic acid in a nucleic acid cassette can be transcribed and when necessary translated in the transfected cells. The term vector as used herein can refer to nucleic acid, e.g., DNA derived from a plasmid, cosmid, phagemid, bacteriophage, virus, retrovirus, adenovirus, adeno-associated virus, lentivirus, or other type of virus into which one or more fragments of nucleic acid may be inserted or cloned which encode for particular proteins. The term “plasmid” as used herein refers to a construction comprised of extrachromosomal genetic material, usually of a circular duplex of DNA which can replicate independently of chromosomal DNA. The plasmid does not necessarily replicate.

Any suitable vectors are envisaged as within the scope of the instant disclosure. The polynucleotides enabling the genetic modifications of the disclosure can be cloned into a number of types of vectors. For example, the polynucleotides enabling the genetic modifications of the disclosure may be cloned into a vector including, but not limited to a plasmid, a phagemid, a phage derivative, an animal virus, and a cosmid. Vectors of particular interest include expression vectors, replication vectors, probe generation vectors, and sequencing vectors. Expression vectors may be provided to cells, such as immune cells, in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; U.S. Pat. No. 6,326,193, and in particular, U.S. Pat. No. 11,065,347, which are incorporated herein by reference in their entireties).

The purpose of the vector is to provide a nucleic acid sequence in cells or tissue. Expression includes the efficient transcription of an inserted gene or nucleic acid sequence. Expression products may be proteins, polypeptides, or RNA. The nucleic acid sequence can be contained in a nucleic acid cassette. Expression of the nucleic acid can be continuous, constitutive, or regulated. The vector can also be used as a prokaryotic element for replication of plasmid in bacteria and selection for maintenance of plasmid in bacteria.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). One method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In some embodiments, transducing comprises either calcium phosphate-mediated gene transfer, DEAE-dextran-mediated gene transfer, liposome-mediated gene transfer, electroporation-mediated gene transfer, viral vector-mediated gene transfer, or nucleofection-mediated gene transfer. In some embodiments, transducing is accomplished by calcium phosphate-mediated gene transfer. In some embodiments, transducing is accomplished by liposome-mediated gene transfer. In some embodiments, transducing is accomplished by electroporation-mediated gene transfer. In some embodiments, transducing is accomplished by viral vector-mediated gene transfer. In some embodiments, transducing is accomplished by nucleofection-mediated gene transfer.

Regardless of the method used to introduce exogenous nucleic acids into a host cell, in order to confirm the presence of the recombinant DNA sequence in the host cell, or confirm effect of genomic modulation, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or other assays.

Genome editing tools such as the clustered regularly interspaced short palindromic repeats (CRISPR) system may be used to genetically modify cells. CRISPR can be used in a wide variety of organisms (e.g., used to add, disrupt, or change the sequence of specific genes). “CRISPR” or “CRISPR gene editing” as used herein refers to a set of clustered regularly interspaced short palindromic repeats, or a system comprising such a set of repeats. “Cas”, as used herein, refers to a CRISPR-associated protein. A “CRISPR/Cas” system refers to a system derived from CRISPR and Cas which can be used to silence, knock out, or mutate a target gene.

The CRISPR/Cas system is based on two elements. The first element is an endonuclease, or Cas, (e.g., Cas9 and MAD7) that has a binding site for the second element, which is the guide polynucleotide (e.g., guide RNA or gRNA). The guide polynucleotide (e.g., guide RNA) directs the Cas protein to double stranded DNA templates based on sequence homology. The Cas protein then cleaves that DNA template. By delivering the Cas protein and appropriate guide polynucleotides (e.g., guide RNAs) into a cell, the organism's genome is cut at a desired location. Following cleavage of a targeted genomic sequence by a Cas/gRNA complex, one of two alternative DNA repair mechanisms can restore chromosomal integrity: 1) non-homologous end joining (NHEJ) which generates insertions and/or deletions of a few base-pairs (bp) of DNA at the gRNA cut site, or 2) homology-directed repair (HDR) which can correct the lesion via an additional “bridging” DNA template that spans the gRNA cut site. CRISPR/Cas systems are classified by class and by type. Class 2 systems currently represent a single interference protein that is categorized into three distinct types (types II, V and VI). Any class 2 CRISPR/Cas system suitable for gene editing, for example a type II, a type V or a type VI system, is envisaged as within the scope of the instant disclosure. Exemplary Class 2 type II CRISPR systems include Cas9, Csn2 and Cas4. Exemplary Class 2, type V CRISPR systems include, Cas12, Cas12a (Cpf1), Cas12b (C2c1), Cas12c (C2c3), Cas12d (CasY), Cas12e (CasX), Cas12f, Cas12g, Cas12h, Cas12i and Cas12k (C2c5). Exemplary Class 2 Type VI systems include Cas13, Cas13a (C2c2) Cas13b, Cas13c and Cas13d.

The CRISPR sequence, sometimes called a CRISPR locus, comprises alternating repeats and spacers. In a naturally-occurring CRISPR, the spacers usually comprise sequences foreign to the bacterium such as a plasmid or phage sequence. As described herein, spacer sequences may also be referred to as “targeting sequences.” In CRISPR/Cas systems for a genetic engineering, the spacers are derived from the target gene sequence (the gNA).

The targeting sequence can be designed or chosen using computer programs known to persons of ordinary skill in the art. The computer program can use variables, such as predicted melting temperature, secondary structure formation, predicted annealing temperature, sequence identity, genomic context, chromatin accessibility, % GC, frequency of genomic occurrence (e.g., of sequences that are identical or are similar but vary in one or more spots as a result of mismatch, insertion or deletion), methylation status, presence of SNPs, and the like. Available computer programs can take as input NCBI gene IDs, official gene symbols, Ensembl Gene IDs, genomic coordinates, or DNA sequences, and create an output file containing sgRNAs targeting the appropriate genomic regions designated as input. The computer program may also provide a summary of statistics and scores indicating on- and off-target binding of the sgRNA for the target gene (Doench et al. Nat Biotechnol. 34:184-191 (2016)).

The target sequence is complementary to, and hybridizes with, the targeting sequence of the gRNA. The target nucleic acid sequence can comprise 20 nucleotides. The target nucleic acid can comprise less than 20 nucleotides. The target nucleic acid can comprise more than 20 nucleotides. The target nucleic acid can comprise at least: 5, 10, 15, 16, 17, 18, 19, 20, 21, 22, 23, 24, 25, 30 or more nucleotides. The target nucleic acid can comprise at most: 5, 10, 15, 16, 17, 18, 19, 20, 21, 22, 23, 24, 25, 30 or more nucleotides.

The CRISPR/Cas system can thus be used to edit a target gene, such as a gene targeted for editing in the cells described herein, by adding or deleting a base pair, introducing a premature stop codon, or introducing a frame-shift mutation which thus decreases expression of the target, in part or completely. The CRISPR/Cas system can alternatively be used like RNA interference, turning off a target gene in a reversible fashion. In a mammalian cell, for example, the RNA can guide the Cas protein to a target gene promoter, sterically blocking RNA polymerases.

In some embodiments, the nucleotide vectors described herein are edited using TALEN gene editing. “TALEN” or “TALEN gene editing” refers to a transcription activator-like effector nuclease, which is an artificial nuclease used to edit a target gene. TALENs are produced artificially by fusing a TAL effector DNA binding domain to a DNA cleavage domain. Transcription activator-like effectors (TALEs) can be engineered to bind any desired DNA sequence, including a portion of target genes such as TCR subunits, MHC class I complex components, or CD52. By combining an engineered TALE with a DNA cleavage domain, a restriction enzyme can be produced which is specific to any desired DNA sequence, including a target gene sequence. These can then be introduced into a cell, wherein they can be used for genome editing. Boch (2011) Nature Biotech. 29: 135-6; and Boch et al. (2009) Science 326: 1509-12; Moscou et al. (2009) Science 326: 3501.

In some embodiments, the nucleotide vectors described herein are edited using ZFN gene editing. “ZFN” or “Zinc Finger Nuclease” or “ZFN gene editing” refer to a zinc finger nuclease, an artificial nuclease which can be used to edit a target gene. Like a TALEN, a ZFN comprises a Fold nuclease domain (or derivative thereof) fused to a DNA-binding domain. In the case of a ZFN, the DNA-binding domain comprises one or more zinc fingers. Carroll et al. (2011) Genetics Society of America 188: 773-782; and Kim et al. (1996) Proc. Natl. Acad. Sci. USA 93: 1156-1160.

A “sequence” of a nucleic acid refers to the order and identity of nucleotides in the nucleic acid. A sequence is typically read in the 5′ to 3′ direction. The terms “identical” or percent “identity” in the context of two or more nucleic acid or polypeptide sequences, refer to two or more sequences or subsequences that are the same or have a specified percentage of amino acid residues or nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997) “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated by reference. Many other optimal alignment algorithms are also known in the art and are optionally utilized to determine percent sequence identity.

Variations in the nucleic acid sequences are contemplated as being encompassed by the present disclosure, provided that the variations in the sequence maintain at least 75%, more preferably at least 80%, 85%, 90%, 95%, and more preferably 99% identity with the 100% sequence identity. Certain percentages in between are included such as 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity. The sequence may be modified for improved therapeutic activity and optimized delivery, for example.

A “label” refers to a moiety attached (covalently or non-covalently), or capable of being attached, to a molecule, which moiety provides or is capable of providing information about the molecule (e.g., descriptive, identifying, etc. information about the molecule) or another molecule with which the labeled molecule interacts (e.g., hybridizes, etc.). Exemplary labels include fluorescent labels (including, e.g., quenchers or absorbers), weakly fluorescent labels, non-fluorescent labels, colorimetric labels, chemiluminescent labels, bioluminescent labels, radioactive labels, mass-modifying groups, antibodies, antigens, biotin, haptens, enzymes (including, e.g., peroxidase, phosphatase, etc.), and the like.

A “linker” refers to a chemical moiety that covalently or non-covalently attaches a compound or substituent group to another moiety, e.g., a nucleic acid, an oligonucleotide probe, a primer nucleic acid, an amplicon, a solid support, or the like. For example, linkers are optionally used to attach oligonucleotide probes to a solid support (e.g., in a linear or other logic probe array). To further illustrate, a linker optionally attaches a label (e.g., a fluorescent dye, a radioisotope, etc.) to an oligonucleotide probe, a primer nucleic acid, or the like. Linkers are typically at least bifunctional chemical moieties and in certain embodiments, they comprise cleavable attachments, which can be cleaved by, e.g., heat, an enzyme, a chemical agent, electromagnetic radiation, etc. to release materials or compounds from, e.g., a solid support. A careful choice of linker allows cleavage to be performed under appropriate conditions compatible with the stability of the compound and assay method. Generally a linker has no specific biological activity other than to, e.g., join chemical species together or to preserve some minimum distance or other spatial relationship between such species. However, the constituents of a linker may be selected to influence some property of the linked chemical species such as three-dimensional conformation, net charge, hydrophobicity, etc. Exemplary linkers include, e.g., oligopeptides, oligonucleotides, oligopoly amides, oligoethyleneglycerols, oligoacrylamides, alkyl chains, or the like. Additional description of linker molecules is provided in, e.g., Hermanson, Bioconjugate Techniques, Elsevier Science (1996), Lyttle et al. (1996) Nucleic Acids Res. 24(14):2793, Shchepino et al. (2001) Nucleosides, Nucleotides, & Nucleic Acids 20:369, Doronina et al (2001) Nucleosides, Nucleotides, & Nucleic Acids 20:1007, Trawick et al. (2001) Bioconjugate Chem. 12:900, Olejnik et al. (1998) Methods in Enzymology 291:135, and Pljevaljcic et al. (2003) J. Am. Chem. Soc. 125(12):3486, all of which are incorporated by reference.

“Fragment” refers to a piece of contiguous nucleic acid that contains fewer nucleotides than the complete nucleic acid.

“Hybridization.” “annealing.” “selectively bind,” or “selective binding” refers to the base-pairing interaction of one nucleic acid with another nucleic acid (typically an antiparallel nucleic acid) that results in formation of a duplex or other higher-ordered structure (i.e. a hybridization complex). The primary interaction between the antiparallel nucleic acid molecules is typically base specific, e.g., A/T and G/C. It is not a requirement that two nucleic acids have 100% complementarity over their full length to achieve hybridization. Nucleic acids hybridize due to a variety of well characterized physio-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes part I chapter 2, “Overview of principles of hybridization and the strategy of nucleic acid probe assays,” (Elsevier, New York), as well as in Ausubel (Ed.) Current Protocols in Molecular Biology, Volumes I, II, and III, 1997, which is incorporated by reference.

The term “attached” or “conjugated” refers to interactions and/or states in which material or compounds are connected or otherwise joined with one another. These interactions and/or states are typically produced by, e.g., covalent bonding, ionic bonding, chemisorption, physisorption, and combinations thereof.

The term “derivative” refers to a chemical substance related structurally to another substance, or a chemical substance that can be made from another substance (i.e., the substance it is derived from), e.g., through chemical or enzymatic modification. To illustrate, oligonucleotide probes are optionally conjugated with biotin or a biotin derivative. To further illustrate, one nucleic acid can be “derived” from another through processes, such as chemical synthesis based on knowledge of the sequence of the other nucleic acid, amplification of the other nucleic acid, or the like.

### Pharmaceutical Compositions

In an aspect, the disclosure provides a pharmaceutical composition comprising the engineered immune cell of the disclosure and one or more pharmaceutically acceptable excipients or diluents.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable diluent or excipient” or “pharmaceutically acceptable carrier” refers to an excipient, diluent, preservative, solubilizer, emulsifier, adjuvant, and/or vehicle with which a nucleic acid of the disclosure, is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens: antioxidants such as ascorbic acid or sodium bisulfite: chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable diluent or excipient” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott. Williams & Wilkins 2003). Except insofar as any conventional media or agent is incompatible with the active compound, such use in the compositions is contemplated.

Formulations of a pharmaceutical composition suitable for administration typically generally comprise the active ingredient combined with a pharmaceutically acceptable diluents or excipients, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. Formulations may also include aqueous solutions which may contain excipients such as salts, carbohydrates and buffering agents or sterile, pyrogen-free, water. Exemplary administration forms may include solutions or suspensions in sterile aqueous solutions, for example, aqueous propylene glycol or dextrose solutions. Such dosage forms can be suitably buffered, if desired.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, and/or aromatic substances and the like which do not deleteriously interact with the formulation. In some embodiments, the pharmaceutical composition comprises said nucleotide vectors in combination with other therapeutically active agents. In some embodiments, the pharmaceutical composition comprises said nucleotide vectors in combination with antibodies specific to a disease cell phenotype. In some embodiments, the disease cell phenotype is that of a malignant cell. In some embodiments, the disease cell phenotype is that of a viral infection.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

### Method of Use

In an aspect, the disclosure provides a method of treating or preventing a disease or disorder in a subject in need thereof, comprising administering the vector for expressing LAMP2 in a cell. In some aspects, the disease or disorder is retinopathy associated with Danon disease. In some aspects, the disease or disorder is age-related macular degeneration.

The terms “subject.” “patient” and “individual” are used interchangeably herein to refer to a vertebrate, preferably a mammal, more preferably a human. Tissues, cells, and their progeny of a biological entity obtained in vivo or cultured in vitro are also encompassed. A “subject,” “patient” or “individual” as used herein, includes any animal that exhibits pain that can be treated with the vectors, compositions, and methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human patients, are included.

In some embodiments, administering comprises administering a therapeutically effective amount to a subject.

As used herein, the term “amount” refers to “an amount effective” or “an effective amount” of a cell to achieve a beneficial or desired prophylactic or therapeutic result, including clinical results. As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

The nucleotide vector may be administered a number of ways depending on whether local or systemic treatment is desired. The nucleotide vector or pharmaceutical compositions are typically suitable for parenteral administration, wherein administration includes any route of administration characterized by physical breaching of a tissue of a subject and administration of the pharmaceutical composition through the breach in the tissue, thus generally resulting in the direct administration into the blood stream, into muscle, or into an organ such as the eye, e.g., injected into the vitreous, suprachoroid or under the retina. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intranasal, intratracheal, intraarterial, intrathecal, intraventricular, intraurethral, intracranial, intratumoral, intraocular, intradermal, and intrasynovial injection or infusions. In some embodiments, the nucleotide vector or pharmaceutical compositions of the present disclosure comprise intravenous administration.

In some embodiments, the nucleotide vector or pharmaceutical composition comprising said nucleotide vector of the disclosure are administered in combination with a combination partner. The term “combination” refers to either a fixed combination in one dosage unit form, or a kit of parts for the combined administration where the engineered immune cell, or pharmaceutical composition comprising said engineered immune cell of the disclosure, and a combination partner (e.g., another drug as explained below, also referred to as “therapeutic agent” or “co-agent”) may be administered independently at the same time or separately within time intervals. In some circumstances the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

### EXAMPLES

### Example 1. Identifying Mechanisms of Retinopathy in Danon Disease

It was hypothesized that LAMP2B deficiency results in Danon retinopathy. More specifically, it was believed that both autophagy and phagocytosis are dysfunctional in hiPSC-RPE from patients caused by LAMP2B deficiency. It was therefore desirable to confirm murine LAMP2 isoform expression in retina and RPE/choroid, before characterizing the phenotype of aged Lamp2 knockout mice (Lamp2y/−) to help clarify the contribution of LAMP2 in retinal degeneration. LAMP2 isoform expression was also tested in a gene therapy construct.

Previous murine studies of cardiac manifestations of Danon disease and Danon disease retinopathy have been highly informative, but do not fully characterize the disease mechanism in human retinal cells due to species and tissue specific differences in LAMP2 isoform expression. For example, studies using cardiomyocytes hiPSC derived from Danon disease patient hiPSCs demonstrated a failure of macroautophagy linked specifically to LAMP2B deficiency, suggesting that RPE integrity is disrupted and results in a reduced electro-oculography response. In contrast, murine retinal studies note decreased phagocytosis as well as increased double membraned autophagic vacuoles. It is therefore necessary to develop a humanized model of Danon disease in order to better understand the disease and provide a platform to test novel treatments.

hiPSC-RPE from Danon disease patients (n=2) and from unaffected control (n=2) hiPSCs were generated and subjected to immunostaining for RPE specific markers RPE65 and Bestrophin, as well as non-specific markers including ZO-1 and MITF. RPE integrity was also tested by measuring transepithelial resistance using an EVOM2 ohmeter, and qRT-PCR was measured to quantify expression of the LAMP2A, 2B and 2C from WT hiPSCRPE lysates.

To test whether autophagy is dysfunctional, LC3-II expression in control hiPSC-RPE was compared with case hiPSC-RPE using immunostaining and western blot using anti-LC3B antibody (Sigma, St. Louis, Mo.). In addition, the cells were stained with periodic acid Schiff to identify the hallmark glycogen filled autophagosomes. Electron microscopy was also performed to examine the number of autophagic vacuoles.

For phagocytosis assays fluorescein isolthiocyanate (FITC) labeled bovine POS (Invision, Seattle, WA) was fed to hiPSC-RPE for 2 hours in normal media. The cells were then washed with PBS and trypan blue before being fixed with 4% PFA for confocal imaging. FITC were then counted. For POS degradation studies, hiPSC-RPE were similarly fed, but the media was changed to normal RPE medium after two hours. The hiPSC-RPE were then similarly fixed to quantify FITC labeled POS 24 hours after cessation of feeding. The objective of this analysis was to establish the main isoforms of LAMP2 found in human RPE, and characterize a humanized in vitro model of Danon disease retinopathy.

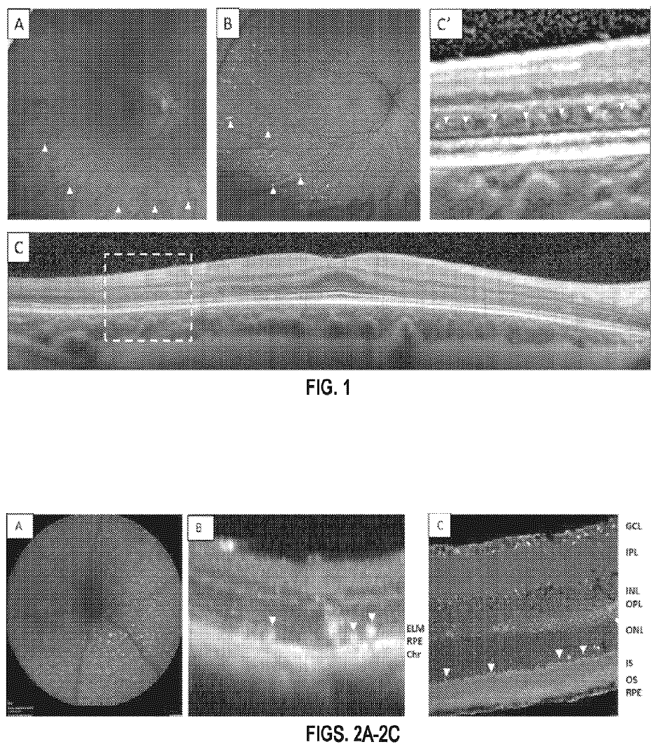

Clinical findings of children (range 6-17 years) with molecularly confirmed Danon disease (n=8) revealed that 7 cases had overt retinopathy with RPE stippling or atrophy and abnormal fundus autofluorescence (FIG. 1, panels A. B). Vision varied from 20/20 to 20/120. Spectral domain optical coherence tomography (SD-OCT) showed ellipsoid zone preservation, but a disruption of the outer nuclear layer (ONL) peripheral to the fovea in all patients (FIG. 1, panel C). Five patients were able to complete ERG testing and of these 3 had markedly reduced ERGs.

Murine studies were also performed used a LAMP2 knockout (KO) mouse model generated on a C57BL/6 background. Findings in 18-22 month old (mo) male LAMP2 KO mice (n=4) were compared with those in 20-24 mo wild type (WT) male mice (n=3). The findings in mice mirrored those seen in children with autofluorescent spots and disruption of the outer retina (FIGS. 2A-2B). The findings suggested that neuroretinal findings may preceded RPE loss.

Immunostaining for LAMP2 was therefore performed and revealed that LAMP2 was expressed not only in the RPE but also in the ganglion cell layer and in the outer nuclear layer (ONL) (FIG. 2C).

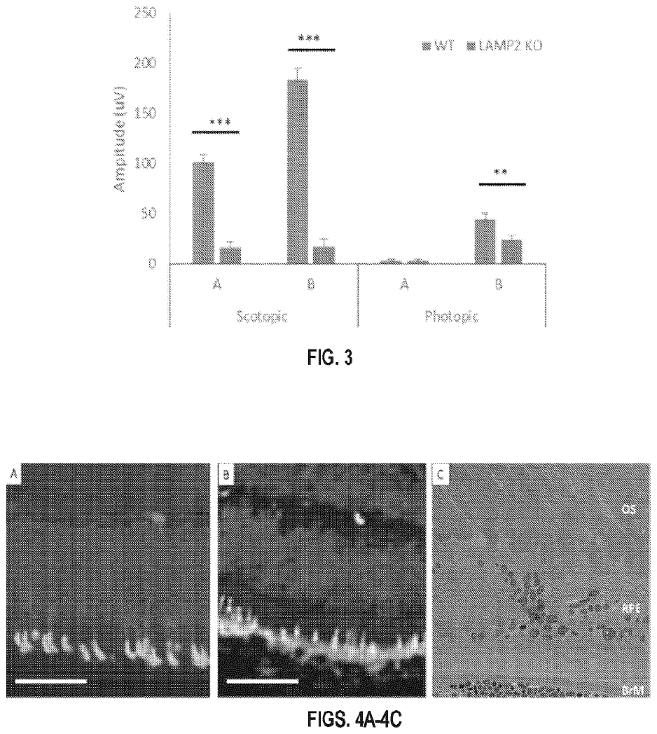

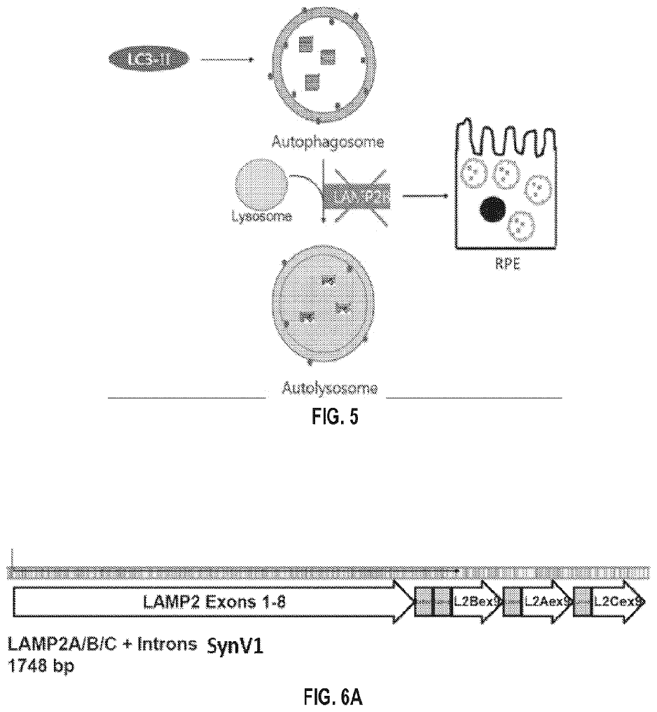

In order to see whether retinal function was affected by LAMP2 KO, electroretinography was performed. The LAMP2 KO mice had a significantly reduced response to stimulation (FIG. 3). Immunostaining for cone receptor opsins and electron microscopy were performed to determine whether the reduced response was due to cell loss or dysfunction (FIG. 4). This analysis demonstrated that, even in aged mice, RPE and photoreceptors were still present. This suggests that the reduced visual response in the model of LAMP2 associated retinopathy was at least partially attributable to dysfunction, and that the neuroretina may also be impacted in vision loss.

### Example 2. Treating Danon Disease Retinopathy with LAMP2 Expression Construct

Currently, there are no treatments for retinopathy associated with Danon disease, however gene therapy for inherited eye disease has developed rapidly in recent years. Gene therapy to replace deficient LAMP2A and LAMP2B in the retina and RPE is therefore likely to provide a therapeutic strategy for treating retinopathy associated with Danon disease. Because LAMP2A and LAMP2B are the likely deficient proteins in the eye, LAMP2A and LAMP2B were delivered by subretinal injection in the eyes of Danon patients using an adeno associated viral vector.

To test whether LAMP2 gene augmentation can reverse the in vitro phenotype in Danon disease patient hiPSC-RPE, rAAV-LAMP2 was added to the media of cells and changed after one day of incubation. Cells were then grown for a further 4 days before being harvested and tested for LAMP2 expression using western blot and qPCR, and for phagocytosis, TER, and autophagic flux.

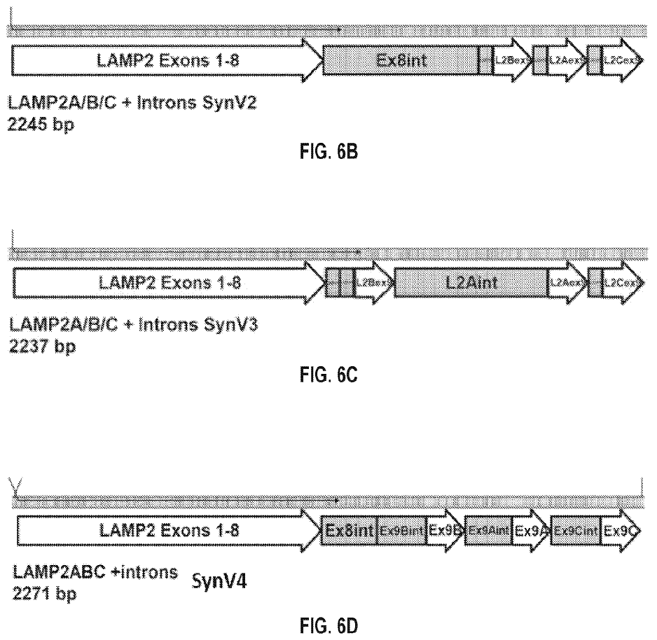

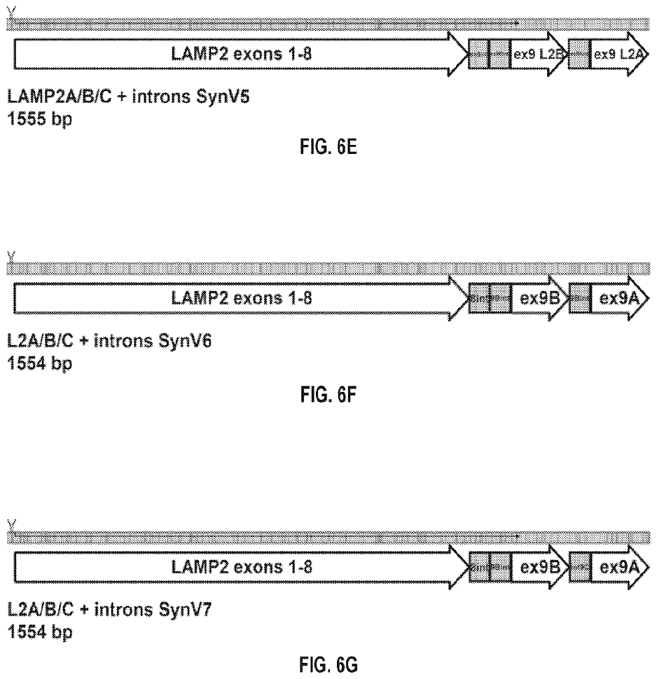

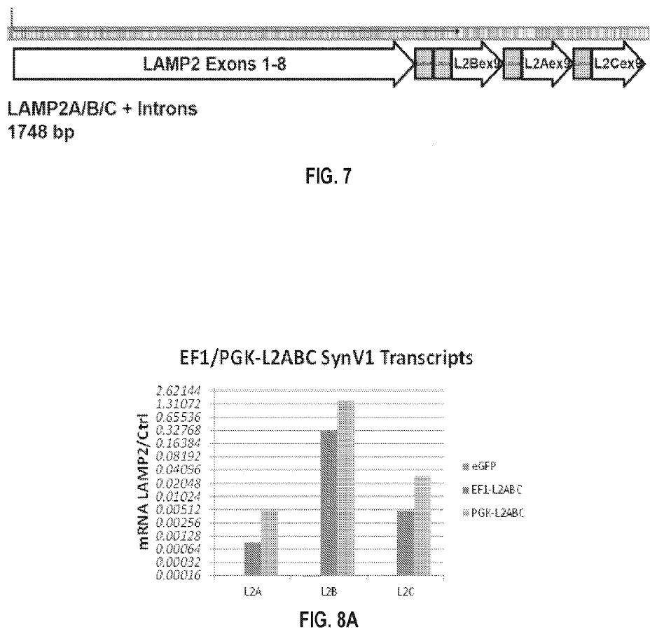

Various embodiments of the LAMP2 gene were constructed with contiguous exons 1-8 and adopted intronic sequences to force alternative acceptor site selection of the LAMP2A/B/C exon 9 (FIGS. 6A-6G, FIG. 7, and SEQ ID Listing). The constructs were designed by computer modeling, ordered, integrated into plasmids and tested on patient hiPSCs. Expression of LAMP2A, 2B and 2C was confirmed using an in vitro disease model.

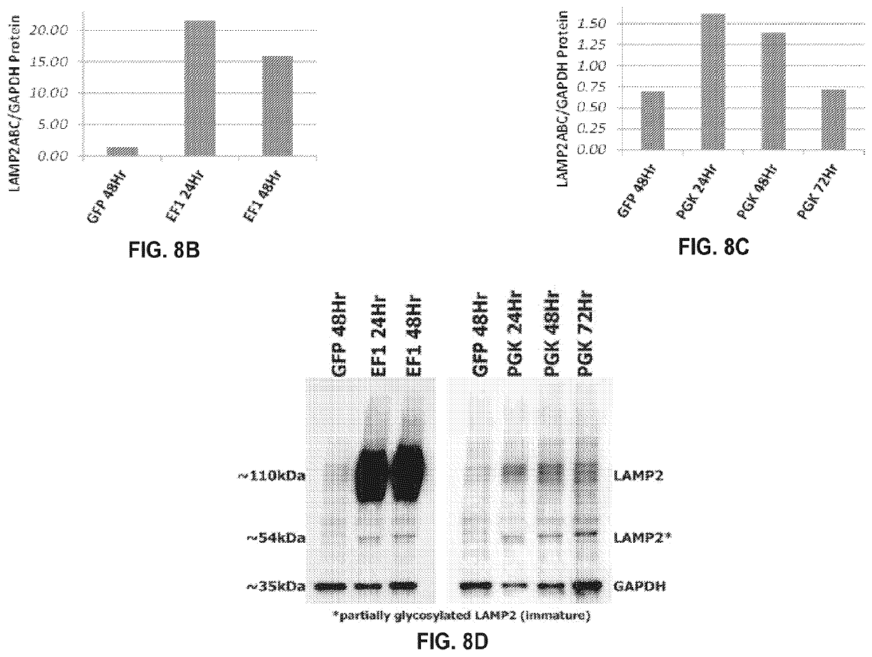

It was hypothesized that incorporating a promoter with the LAMP2 construct would be effective to increase LAMP2 expression activity in the cells. To test expression, the constructs carrying promoters were inserted into a plasmid, and LAMP2 expression was measured. Two promoters in particular were tested, EF1 and PGK (FIGS. 8A-8D). See, Uetsuki et al., J. Biol. Chem. 264, 10, 5791-5796 (1989) for EF1 promoter background, and Singer-Sam et al., Gene 32, 3, 409-417 (1984) for PGK promoter background. Both promoters were shown to significantly increase LAMP2 expression as compared to the construct without the promoter (FIG. 8A). Comparatively, the PGK promoter yields about 10-20× less mRNA per unit time than the EF1 promoter. However, the EF1 promoter may generate too much protein and be toxic to cells. Thus, the PGK promoter may be more preferable as a slow and steady promoter for delivery to the choroid and retina.

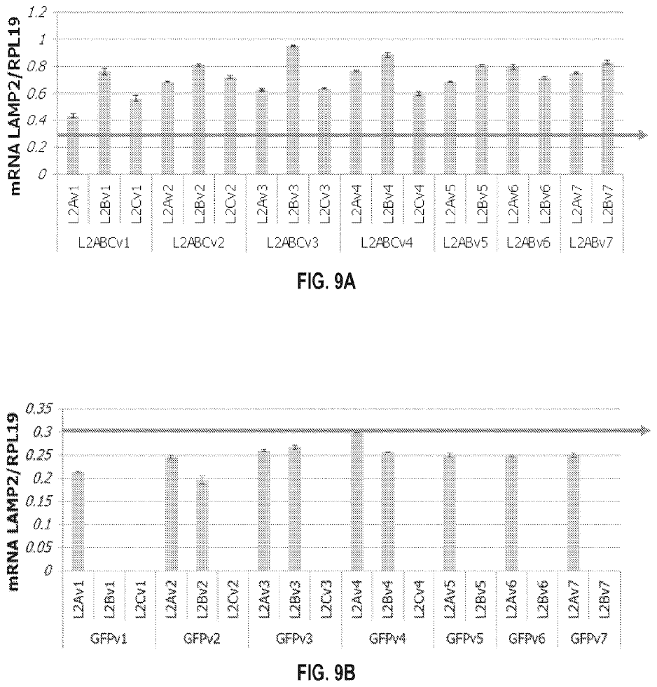

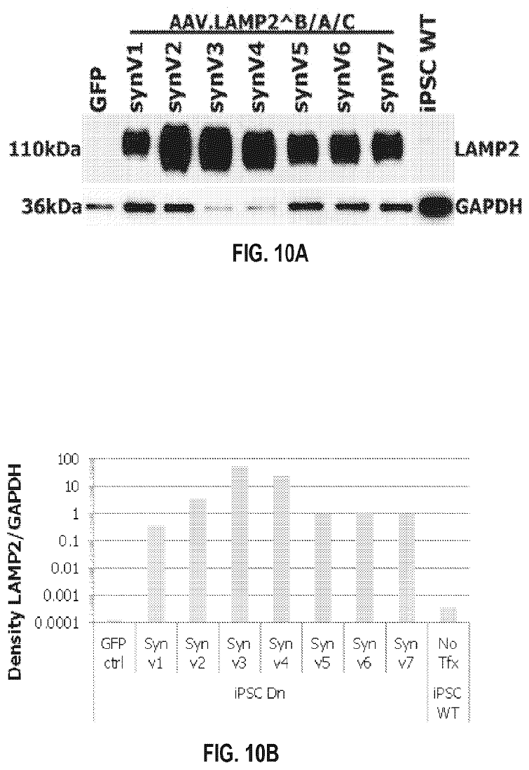

Additional analysis was performed on the EF1 promoter constructs to better observe and quantify the increased expression of the EF1-LAMP2ABC (LAMP2ABCplusIntrons_Gblock_SynV1 (SEQ ID NO: 1)) construct as compared to a previous LAMP2ABC construct (FIGS. 9A-9B). The latter construct only encodes LAMP2B and contains the CAG promoter (see, U.S. Pat. No. 11,065,347). The constructs listed in FIG. 9A correspond to SEQ ID NOs: 1-7 herein, respectively. FIG. 9B is a control experiment transfected with a plasmid encoding only GFP. These cells do not express any LAMP2 protein but express some endogenous LAMP2 transcript but in very low abundance relative to transfected levels. Thus, FIG. 9B illustrates what is expected in a typical qPCR analysis in the absence of an engineered LAMP2ABC or LAMP2AB AAV plasmid. Note that the horizontal arrowed line in FIGS. 9A and 9B mark the cut-off value for endogenous detection versus ectopic transcript production. First, sequential qPCR was performed, which demonstrated ectopic expression of AAV.L2ABC in Danon iPSCs when subjected to the EF1-LAMP2ABC construct (FIG. 9A) as compared to the conventional LAMP2ABC construct (FIG. 9B). This finding was affirmed via a western blot protein quantification analysis (FIG. 10A).

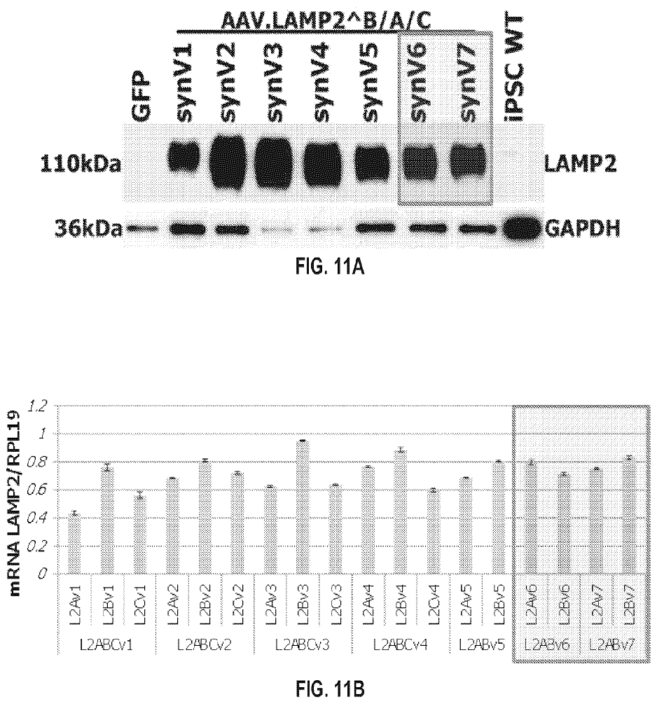

During preliminary testing of the LAMP2 constructs, varying levels of LAMP2 expression were observed, both with respect to LAMP2A vs. LAMP2B and across the different constructs. Notably, LAMP2ABCplusIntrons_Gblock_SynV6 (SEQ ID NO:6) and LAMP2ABCplusIntrons_Gblock_SynV7 (SEQ ID NO:7) resulted in consistent expression in LAMP2A vs. LAMP2B, and the expression levels of each construct were also similar (FIG. 11B). Because expression levels for SEQ ID NO:6 were more consistent for LAMP2A vs. LAMP2B, SEQ ID NO:6 was selected for further testing and optimization.

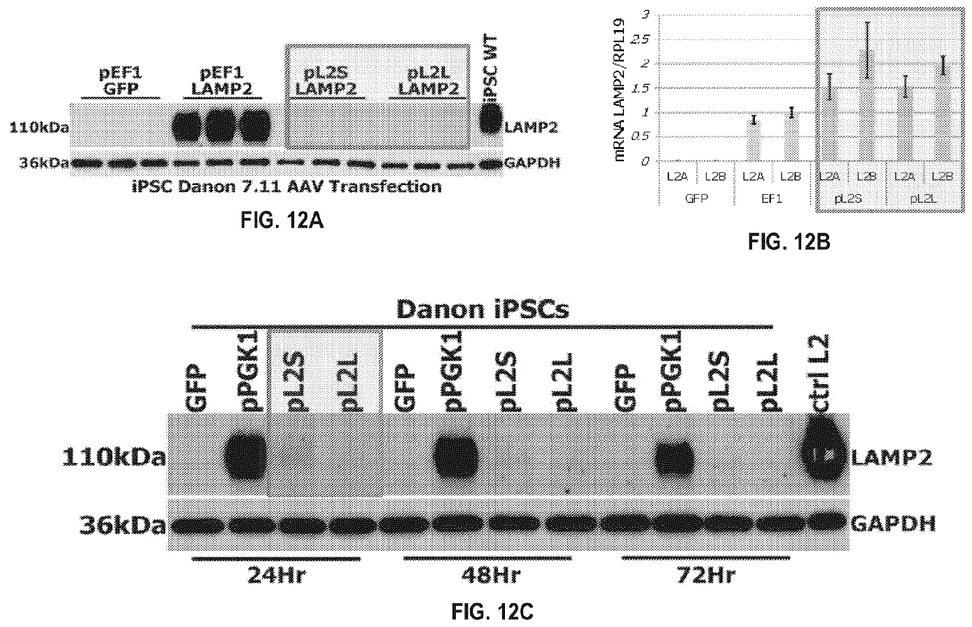

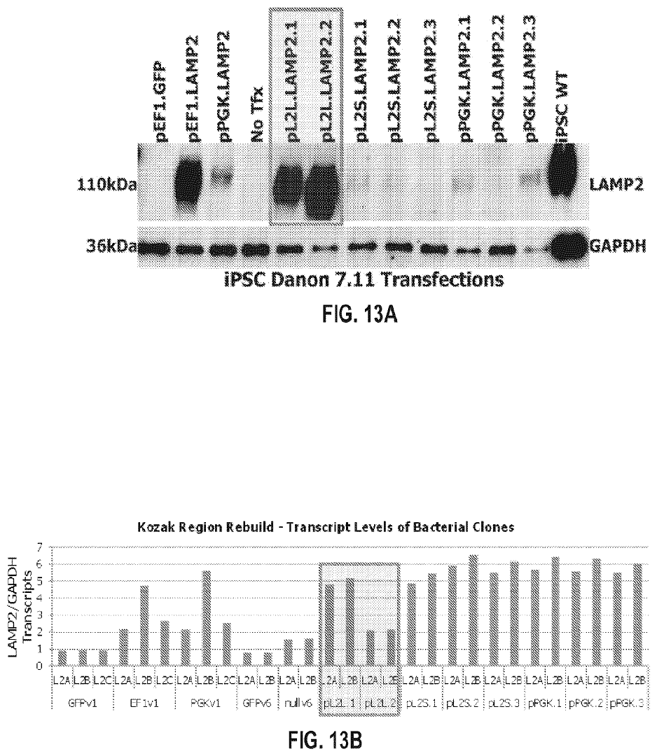

In particular, SEQ ID NO: 6 was paired with various promoters (including EF1 and PGK) and the relative protein expression levels were tested at various time periods (FIGS. 12-13). As previously observed, EF1 and PGK demonstrated increased LAMP2 expression. Additional promoters L2S and L2L were also tested, and in the western blot analysis, did not appear to demonstrate increased LAMP2 expression as compared to the control. However, the quantified transcription of the L2S and L2L promoter constructs indicated significant increase in LAMP2A and LAMP2B transcription, far surpassing the levels observed for the EF1 constructs. L2S and L2L would therefore be optimal promoters, because each promoter increases transcription without elevating protein concentration within the cell, thereby reducing the chance of cell death. This observation indicates that protein abundance post-injection is not necessarily indicative of transcription activity in the cell. It also indicates that the ribosomal targeting may also be an important cellular mechanism in the context of LAMP2 deficiency and expression.

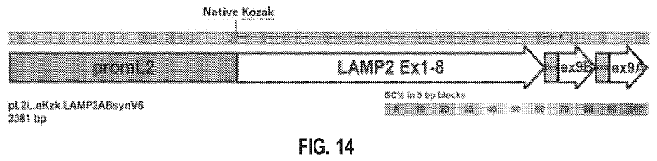

An exemplary L2L-LAMPABsynV6 construct is shown in FIG. 14 and provided as SEQ ID NO: 8. Collectively, L2S and L2L promoters are contained in SEQ ID NO: 8. L2S (LAMP2 short) is an abbreviated L2L (LAMP2 long) promoter and is truncated to approximately 700 bp five prime of the transcriptional start site.

For use as a treatment, the vector may be injected into the vitreous, suprachoroid or under the retina in patients with retinal LAMP2 deficiency. The vector will then transduce retinal cells, primarily, but not limited to, photoreceptors and retinal pigment epithelial cells. The cells will then start expressing LAMP2 isoforms. This should restore function to the cells, preserve and in some cases improve visual function.

It will be understood from the foregoing description that various modifications and changes may be made in the various embodiments of the present disclosure without departing from their true spirit. The description provided herein is intended for purposes of illustration only and is not intended to be construed in a limiting sense. Thus, while the presently disclosed inventive concepts have been described herein in connection with certain embodiments so that aspects thereof may be more fully understood and appreciated, it is not intended that the presently disclosed inventive concepts be limited to these particular embodiments. On the contrary, it is intended that all alternatives, modifications and equivalents are included within the scope of the presently disclosed inventive concepts as defined herein. Thus the examples described above, which include particular embodiments, will serve to illustrate the practice of the presently disclosed inventive concepts, it being understood that the particulars shown are by way of example and for purposes of illustrative discussion of particular embodiments of the presently disclosed inventive concepts only and are presented in the cause of providing what is believed to be a useful and readily understood description of procedures as well as of the principles and conceptual aspects of the inventive concepts. Changes may be made in the construction and formulation of the various components and compositions described herein, the methods described herein or in the steps or the sequence of steps of the methods described herein without departing from the spirit and scope of the presently disclosed inventive concepts.

### ADDITIONAL REFERENCES

Taylor M R G, Adler E D. Danon Disease. Gene Rev. NCBI Bookshelf. 2020; Available from: https://www.ncbi.nlm.nih.gov/books/NBK554742/.
Brambatti M, Caspi O, Maolo A, Koshi E, Greenberg B, Taylor M R G, Adler E D. Danon disease: Gender differences in presentation and outcomes. Int J Cardiol. 2019; 286:92-98. Available from: www.ncbi.nlm.nih.gov/pubmed/30857840.
Notomi S, Ishihara K, Efstathiou N E, et al. Genetic LAMP2 deficiency accelerates the age-associated formation of basal laminar deposits in the retina. Proc Natl Acad Sci USA. 2019; 116(47):23724-23734. doi:10.1073/pnas.1906643116.
Greenberg B, Eshraghian E, Battiprolu P, Ricks D, Yarabe P, Schwartz J, Patel K, Shah G, Trevejo J. Results from First-In-Human Clinical Trial of RP-A501 (AAV9:LAMP2B) gene therapy treatment for Danon disease. Circulation. 2021 Nov. 16: 144(Suppl_1):A10727.

What is claimed is:

1. A method for treating a disease or condition characterized by LAMP2 deficiency in a patient, the method comprising administering to the patient an effective amount of a nucleotide vector for expressing two or more lysosome associated membrane protein 2 (LAMP2) isoforms in a cell of the patient.

2. The method of claim 1, wherein the disease or condition characterized by LAMP2 deficiency is a retinal disease.

3. The method of claim 1, wherein the two or more LAMP2 isoforms are selected from LAMP2A, LAMP2B and LAMP2C isoforms.

4. The method of claim 1, wherein the vector is an adeno-associated virus expressing LAMP2 in a cell.

5. The method of claim 2, wherein the cell is selected from a neuroretinal cell and a retinal pigment epithelium (RPE) cell.

6. The method of claim 2, wherein the vector is administered by injection into the vitreous, the suprachoroid or under the retina of the patient.

7. The method of claim 2, wherein the method restores retinal function in the patient.

8. The method of claim 2, wherein the method preserves or improves vision in the patient.

9. The method of claim 2, wherein the retinal disease is Danon disease.

10. The method of claim 2, wherein the retinal disease is age-related macular degeneration.

11. The method of claim 1, wherein the LAMP2 isoforms comprise LAMP2A and LAMP2B isoforms.

12. A composition for treating a disease or condition characterized by LAMP2 deficiency in a patient, the composition comprising a nucleotide vector encoding two or more lysosome associated membrane protein 2 (LAMP2) isoforms selected from LAMP2A, LAMP2B and LAMP2C isoforms.

13. The composition of claim 12, wherein the disease or condition characterized by LAMP2 deficiency is retinal disease.

14. The composition of claim 13, wherein the vector is an adeno-associated virus.

15. The composition of claim 13, wherein the composition targets neuroretinal and retinal pigment epithelium (RPE) cells to express LAMP2.

16. The composition of claim 13, wherein the composition restores retinal function in the patient.

17. The composition of claim 13, wherein the composition preserves or improves vision in the patient.

18. The composition of claim 13, wherein the retinal disease is Danon disease or age-related macular degeneration.

19. The composition of claim 12, wherein the LAMP2 isoforms comprise LAMP2A and LAMP2B isoforms.

20. A neuroretinal cell or retinal pigment epithelium (RPE) cell modified with a nucleotide vector encoding two or more lysosome associated membrane protein 2 (LAMP2) isoforms.
